(19) United States
(12) Patent Application Publication
(10) Pub. No.: US 2007/0093559 A1
(43) Pub. Date: Apr. 26, 2007

(54) Catalytic reduction and oxidation processes

(76) Inventors: Jens K. Norskov, Holte (DK); Jens H. Hyldtoft, Kokkedal (DK); Bjerne S. Clausen, Vedbaek (DK); Soren Dahl, Hillerod (DK)

Correspondence Address: DICKSTEIN SHAPIRO LLP, 1825 EYE STREET NW, Washington, DC, 20006-5403, US

(21) Appl. No.: 10/567,149

(22) PCT Filed: Sep. 10, 2004
(86) PCT No.: PCT/EP04/08957

§ 371 Date: Aug. 23, 2006

(30) Foreign Application Priority Data
Aug. 13, 2003 (DK) PA 2003 01158

(51) Int. Cl. C07C 27/26; C01B 15/14; B01J 23/58

(52) U.S. Cl. 518/726; 502/330; 423/325

(57) ABSTRACT

Catalytic process for oxidation or reduction of organic and inorganic compounds comprising contacting the compound under oxidation or reduction conditions with a supported catalyst consisting of nickel as the active catalytic component promoted with silver or gold, the silver or gold being present in an amount between 0.001% to 30% by weight calculated on the amount of nickel in the catalyst.

The present invention is related to catalytic reduction and oxidation processes and a catalyst for use in the process. In particular, the invention involves a silver or gold promoted nickel catalyst for use in different catalytic reduction and oxidation reactions.

Nickel is a well-known catalyst for a number of reactions. A major problem in several of these reactions is the high reactivity of nickel. For hydrocarbon reactions this leads to deposition of carbonaceous species on the surface destroying the activity under certain conditions [J. R. Rostrup-Nielsen, Steam Reforming Catalysts, Danish Technical Press Inc., Copenhagen 1975] and for oxidation reactions nickel oxides are often formed. Nickel catalysts are also found in some cases to form too many by-products, i.e. the selectivity is low compared to that of for instance platinum.

Examples of reduction and oxidation reactions that are problematic when catalysed by a nickel catalyst and where for instance a noble metal based catalyst presents fewer problems are:

Hydrogenation and dehydrogenation reactions:
CₓH_{y}+H₂ [symbol] CₓH_{y+2}

Selective oxidation of, for instance, CO in a hydrogen atmosphere without simultaneous oxidation of hydrogen:
2CO+O₂→2CO₂

Oxidation of SO_{2:}
2SO₂+O₂→2SO₃

Reduction of NO with CO:
2NO+2CO→N₂+2CO₂

CO methanisation:
CO+3H₂→CH₄+H₂O

Ethane hydrogenolysis:
C₂H₆+H₂→2CH₄

The usual alternative in cases like this is to use a noble metal based catalyst of for instance platinum or palladium. These materials are less reactive and therefore present fewer problems in terms of surface contamination. Platinum and palladium are, however, considerably more expensive than nickel, and it is therefore highly desirable to be able to modify the surface properties of nickel such that it exhibits a reactivity similar to for instance platinum or palladium. In other words, that nickel shows a more “noble” behaviour. This would open up the possibility of a cheaper catalyst for a number of reactions.

Nickel catalysts promoted with silver and therefore expected to exhibit some “noble” behaviour are known for other purposes, see for instance U.S. Pat. No. 4,060,498.

It is an objective of the invention to modify nickel catalysts in order to change their reactivity and provide them with a more “noble” behaviour thereby rendering the catalysts more suitable than pure nickel catalysts for a number of oxidation and reduction reactions.

Accordingly, a catalyst consisting of nickel as the active catalytic component promoted with silver or gold is provided, the silver or gold being present in an amount between 0.001% to 30% by weight calculated on the amount of nickel in the catalyst for use in a catalytic process for oxidation or reduction of organic and inorganic compounds.

Based on the above observations, a broad embodiment of the invention is directed towards a method for modifying a nickel catalyst by promotion with silver or gold, thereby changing its catalytic properties such that it becomes more “noble” in behaviour and having properties comparable to for instance those of platinum.

In some reduction and oxidation reactions a higher conversion or selectivity than obtained with pure nickel is required, while in other reactions a reduced conversion or selectivity when compared to pure nickel is desired.

This can be accomplished by using the silver or gold promoted nickel catalyst according to the invention. Depending on the type of reaction, the reactivity can either show an increase or a decrease. This is due to the fact that the promoter atoms present block certain types of active sites on the catalyst. Not all the active sites are blocked. This phenomenon can also be observed in other systems, e.g. ruthenium decorated with gold (S. Dahl et. al., Phys. Rev. Letters, 83 (1999) 1814). For reactions or conditions where poisoning is important, the blocking of poisonous side reactions will give increased activity. For reactions or conditions where such side reactions are of minor importance, a reduction in the reactivity will be observed since fewer active sites will be available due to the deposition of the promoter atoms. Platinum is characterized by having few side reactions in the mentioned reactions and therefore the promoted nickel catalyst will show properties comparable to those of platinum.

The amount of silver or gold to be incorporated in the catalyst depends on the nickel edge surface area. The silver or gold promoted nickel catalyst can be prepared by co-impregnation or sequential impregnation of the carrier material with solutions containing a soluble nickel salt and a salt of the silver or gold promoter.

In order to modify the properties of nickel with silver or gold, the silver or gold must be positioned at the nickel surface. In some cases this can be achieved by co-impregnation of a carrier with a solution containing both a nickel and a silver precursor, as for instance mentioned in U.S. Pat. No. 4,060,498. However, this patent only covers catalysts for steam reforming.

Another way of achieving a partly covered nickel surface is by sequential impregnation, where the carrier is first impregnated with a nickel precursor, calcined and reduced and then impregnated with a silver precursor. Silver will then be placed at the nickel surface either by reduction by nickel surface atoms or with the aid of a reductant added during the deposition. Such processes are further described by J. Margitfalvi, S. Szabo, F. Nagy in Supported Bimetallic Catalysts prepared by Controlled Surface Reactions, Studies in Surface Science and Catalysis, Vol. 27, chapter 11, Elsevier 1986. The deposition of gold should be done in an analogous manner, as mentioned in U.S. Pat. No. 5,997,835, which, however, also covers only steam reforming.

Suitable precursors are salts including chlorides, nitrates, carbonates, acetates or oxalates.

Yet another way of ensuring silver or gold deposition at the nickel surface is to use chemical vapour deposition of silver or gold at the reduced nickel catalyst. Suitable precursors include silver (I)-(β-diketonato) complexes for silver and gold (III)-(β-diketonato) complexes for gold.

Carrier materials are conventionally selected from the group of carbon, alumina, magnesia, titania, silica, zirconia, beryllia, thoria, lanthania, calcium oxide and compounds or mixtures thereof. Preferred materials comprise alumina, titania and magnesium aluminum spinel.

The promoted Ni catalyst, thus obtained, can be used in different catalytic reactions as mentioned earlier and exemplified in the following, thus substituting the more expensive platinum catalysts.

The invention will further be described in the following examples. All concentrations of nickel and silver or gold in the catalysts are given in weight % (wt %).

### EXAMPLE 1

### Benzene Hydrogenation

A silver promoted nickel catalyst consisting of 17% by weight of nickel and 0.3% by weight of silver were prepared by sequential impregnations of an MgAl₂O₄ spinel carrier with nickel nitrate followed by silver nitrate. Before the impregnation with the silver precursor, the nickel nitrate was decomposed. After drying, the catalyst pellets were loaded in a reactor and activated by heating to 500° C. in flowing hydrogen at atmospheric pressure. The amount of by-products produced when converting benzene to cyclohexane was determined under the following conditions:

| | |
|---|---|
| Catalyst size, μm | 150-300 |
| Catalyst amount, g | 0.1 |
| Inert size, μm | 150-300 |
| Inert amount, g | 0.1 |
| Temperature, ° C. | 300 |
| Pressure, barg | 11 |
| Feed gas composition, Nl/h | |
| H₂ | 6 |
| Benzene as gas | 0.6 |

The conversion of benzene and the yield of by-products calculated on a carbon basis are shown in the Table.

TABLE 1

| | | Benzene | Yield of by- |
|---|---|---|---|
| Catalyst | Carrier | conversion | products |
| 17 wt % Ni | MgAl₂O₄ | 96.3% | 4.0% |
| 17 wt % Ni/0.3 wt % Ag | MgAl₂O₄ | 97.3% | 2.1% |

The results shown in the Table show a marked decrease in by-products formation for the silver promoted nickel catalyst and a higher conversion.

### EXAMPLE 2

### Preferential CO Oxidation

A silver promoted nickel catalyst sample consisting of 17 wt % nickel and 2.455 wt % silver was prepared as in Example 1. After drying, the catalyst pellets were loaded in a reactor and activated during heating to 570° C. in flowing hydrogen at atmospheric pressure. The activity for CO and H₂ oxidation was determined under the following conditions:

| | |
|---|---|
| Catalyst size, μm | 150-300 |
| Catalyst amount, mg | 50 |
| Temperature, ° C. | 60 |
| Total flow rate, Nl/h | 1.8 |
| Feed gas composition, vol % | |
| CO | 0.5 |
| O₂ | 0.5 |
| H₂ | 4.4 |
| Ar | 94.6 |

The conversions to CO₂ and H₂O for a pure Ni and an Ag promoted Ni catalyst are shown in Table 2.

TABLE 2

| | | Conversion for: | |
|---|---|---|---|
| Catalyst | Carrier | CO + O₂ → CO₂ | H₂ + O₂ → H₂O |
| 17 wt % Ni | MgAl₂O₄ | <0.1% | Zero |
| 17 wt % Ni/ | MgAl₂O₄ | 2.23% | <0.4% |
| 2.455 wt % | | | |
| Ag | | | |

As seen from Table 2, the nickel catalyst is nearly inactive for CO oxidation and inactive for H₂ oxidation. Modifying the nickel catalyst with silver enhances the reactivity in accordance with the invention and enables the preferential oxidation of CO to CO₂.

For instance the catalyst can be used in the clean up by reformate gas used as fuel for a PEM fuel cell.

### EXAMPLE 3

### SO₂ Oxidation

A silver promoted nickel catalyst sample consisting of 4 wt % nickel and 0.2 wt % silver was prepared as in Example 1 by sequential impregnations of a titania (TiO₂) carrier. A gold promoted nickel catalyst sample consisting of 17 wt % nickel and 0.3 wt % gold on a spinel carrier was prepared as in Example 1 using [Au(NH₃)₄] (NO₃)₃ as Au precursor. After drying, the catalyst pellets were loaded in a reactor and the activity for SO₂ oxidation measured under the following conditions at atmospheric pressure:

| Catalyst size, mm × mm | 9 × 3 for the TiO₂ carrier |
|---|---|
| | 4.5 × 4.5 for the MgAl₂O₄ carrier |
| Catalyst amount, g | 1.66-4.75 |
| Temperature, ° C. | 380 |
| Feed gas composition, Nl/h | |
| SO₂ | 0.7 |
| O₂ | 7 |
| N₂ | 92.3 |

The activities are shown in Table 3.

TABLE 3

| Catalyst | Carrier | Relative activity |
|---|---|---|
| 4 wt % Ni | TiO₂ | 100 |
| 4 wt % Ni/ | TiO₂ | 700 |
| 0.2 wt % Ag | | |
| 16 wt % Ni | MgAl₂O₄ | 100 |
| 16 wt % Ni/ | MgAl₂O₄ | 200 |
| 0.3 wt % Au | | |

As seen from the Table, there is a marked improvement in oxidation activity for both the silver promoted nickel catalyst and the gold promoted nickel catalyst compared to the pure nickel catalyst in accordance with the invention.

### EXAMPLE 4

### NO Reduction

A silver promoted nickel catalyst sample containing 16% by weight of nickel and 0.577 wt % silver was prepared as in Example 1. After drying, the catalyst pellets were loaded in a reactor and activated during heating to 500° C. in flowing hydrogen at atmospheric pressure. The activity for reduction of NO with CO was determined under the following conditions:

| | |
|---|---|
| Catalyst size, μm | 150-300 |
| Catalyst amount, g | 0.5 |
| Temperature, ° C. | 200 |
| Total flow rate | 2.4 Nl/h |
| Feed gas composition, vol ppm | |
| CO | 2850 vol ppm |
| NO | 2850 vol ppm |
| Balance | He |

The conversion of NO for the catalysts is shown in Table 4.

TABLE 4

| Catalyst | Carrier | Relative conversion |
|---|---|---|
| 16 wt % Ni/ | MgAl₂O₄ | 100 |
| 16 wt % Ni/0.577 wt % | MgAl₂O₄ | 150 |
| Ag | | |

As seen from Table 4, the activity of the nickel catalyst is markedly improved by promoting with silver in accordance with the invention. This could for instance be used as a gasoline exhaust catalyst.

### EXAMPLE 5

### CO Methanisation

A silver promoted nickel catalyst sample consisting of 17 wt % nickel and 3 wt % silver was prepared as in Example 1.

After drying, the catalyst pellets were loaded in a reactor and activated during heating to 500° C. in flowing hydrogen at atmospheric pressure. The activity for CO methanisation was determined at atmospheric pressure under the following conditions:

| | |
|---|---|
| Catalyst size, μm | 150-300 |
| Catalyst amount, g | 0.1 |
| Temperature, ° C. | 250 |
| Feed gas composition, Nl/h | |
| CO | 0.13 |
| H₂ | 13.0 |

The activities are shown in Table 5.

TABLE 5

| Catalyst | Carrier | Relative activity |
|---|---|---|
| 16 wt % Ni | MgAl₂O₄ | 100 |
| 16 wt % Ni/3 wt % Ag | MgAl₂O₄ | 50 |

As apparent from Table 5 the reactivity for conversion of CO to CH₄ is strongly reduced by modifying the nickel containing catalyst with silver in accordance with the invention.

The catalyst can be used in reactions where CO methanisation is undesirable as in for instance methanol synthesis and water gas shift reactions.

### EXAMPLE 6

### Ethane Hydrogenolysis

A silver promoted nickel catalyst sample consisting of 0.9 wt % nickel and 0.1 wt % silver was prepared by co-impregnation with nickel and silver nitrate on a spinel carrier together with a nickel containing catalyst (1 wt %). After drying, the catalyst pellets were loaded in a reactor and activated during heating to 500° C. in flowing hydrogen at atmospheric pressure. The activity for ethane hydrogenolysis was determined at atmospheric pressure under the following conditions:

| | |
|---|---|
| Catalyst size, μm | 150-300 |
| Catalyst amount, g | 0.1 |
| Temperature, ° C. | 325 |
| Feed gas composition, Nl/h | |
| C₂H₆ | 0.12-0.21 |
| H₂ | 0.90-1.80 |
| He | 4.0-4.1 |

The Kinetic Expression:
Reaction Rate=k·P(ethane)·(P(hydrogen))^{-0.5}

where P(x) is the pressure of component x was found to give an excellent description of experimental results. The activities expressed by rate constants, k, are shown in Table 6.

TABLE 6

| | | Rate Constant |
|---|---|---|
| Catalyst | Carrier | μmol/(g s (bar)^{0.5}) |
| 1 wt % Ni | MgAl₂O₄ | 133 |
| 0.9 wt % Ni/0.1 wt % Ag | MgAl₂O₄ | 12 |

As apparent from Table 6 the reactivity for conversion of C₂H₆ to CH₄ is strongly reduced by modifying the nickel containing catalyst with silver in accordance with the invention.

The catalyst can be used in reactions where C—C bond hydrogenolysis is undesirable, e.g. in hydrogenation reactions as decribed in Example 1.

1. Catalytic process for oxidation or reduction of organic and inorganic compounds comprising contacting the organic or inorganic compound under oxidation or reduction conditions with a supported catalyst consisting of nickel as the active catalytic component promoted with silver or gold, the silver or gold being present in an amount between 0.001% to 30% by weight calculated on the amount of nickel in the catalyst.

2. Process according to claim 1, wherein the catalytic process is dehydrogenation.

3. Process according to claim 1, wherein the catalytic process is SO₂ oxidation.

4. Process according to claim 1, wherein the catalytic process is NO reduction with CO.

5. Process according to claim 1, wherein the catalytic process is CO methanisation.

6. Process according to claim 1, wherein the catalytic process is hydrogenation.

7. Process according, to claim 1, Wherein the catalytic process is ethane hydrogenolysis.

8. Process according to claim 1, wherein the support is alumina, titania or magnesium aluminium spinel.
